(19) United States
(12) Patent Application Publication
(10) Pub. No.: US 2016/0324776 A1
(43) Pub. Date: Nov. 10, 2016

(54) Cannabinoid caffeinated drinks, powder, beans, and cannabinoid loose tea leaf

(71) Applicant: HDDC Holdings, LLC, Poway, CA (US)

(72) Inventor: Khanh T. Glatzel, Escondido, CA (US)

(73) Assignee: HDDC Holdings, LLC, Poway, CA (US)

(21) Appl. No.: 14/706,736

(22) Filed: May 7, 2015

(51) Int. Cl. A61K 9/00; A23F 5/40; A23F 3/16; A61K 47/48; A23L 2/52; A61K 31/05; A61K 31/352; A23F 5/24; A23F 3/14

(52) U.S. Cl. CPC A61K 9/0095; A23F 5/243; A23F 5/40; A23F 3/163; A23F 3/14; A23L 2/52; A61K 31/05; A61K 31/352; A61K 47/48969; A23V 2002/00

(57) ABSTRACT

This application discloses a caffeinated drink with cannabinoids, wherein the drink retains its original taste and appearance. This application also discloses coffee powder with cannabinoids, roasted coffee beans with cannabinoids, and loose tea leaf with cannabinoids. Methods to the make and use of the above caffeinated drinks, coffee powder, coffee beans, and loose tea leaf are also disclosed.

### CROSS REFERENCE TO RELATED APPLICATIONS

This application claims the benefit under 35 U.S.C. §119(e) of U.S. Provisional Application No. 61/991,968, filed May 12, 2014 and U.S. Provisional Application No. 62/025,832, filed July 17, 2014.

### FIELD OF THE INVENTION

This application generally relates to caffeinated drinks with cannabinoid content incorporated into the drink. This application also relates to methods of making this cannabinoid caffeinated drink, including post brew addition, pre-brew addition after grinding or into loose leaf tea material, and at the roasting or curing stage. This application also relates tea leaf preparations packaged in tea bags and provided in loose leaf form. Consumption of this cannabinoid coffee or tea drink gives the consumer a calm but alert feeling, which is greatly beneficial for concentration and highly intellectual activities.

### BACKGROUND OF THE INVENTION

Coffee is one of the most consumed beverages in the world. It comes from coffee plants, which produce berries that are harvested and dried in order to yield the seeds inside the berries. These seeds are then roasted for a certain amount of time, depending on the desired taste, ground up, and brewed to create coffee. It is estimated that coffee plants are grown in at least 70 countries worldwide. In fact, it is the main agricultural export product in 12 countries, and was the world's seventh-largest legal agricultural export by value in 2005.

Coffee was first cultivated and traded by the Arabs as early as the fifteenth century. Yemen was the first place in Arabia that coffee was cultivated and it quickly spread to Persia, Egypt, Syria, and Turkey. Coffee was consumed in public coffee houses as well as in the home. People frequented the coffee houses for its many social activities, as well as to consume the drink. Coffee eventually made it way to Europe in the seventeenth century and quickly gained widespread popularity. From Europe, coffee was brought to the Americas where huge plantations were established to cultivate the beans.

The consumption of coffee has long held many societal benefits as well. People in Arabia gathered at coffeehouses to discuss many intellectual topics, including politics. A famous coffeehouse in Paris was the meeting place for the leading figures in the Enlightenment. John Adams and Paul Revere plotted the American Revolution against the British from the Green Dragon, a coffeehouse in Boston.

Coffee has become a huge industry worldwide. Starbucks operates 16,600 stores in 40 different countries. Indeed, over 2.25 billion cups of coffee are consumed each day.

Coffee contains caffeine, which is a mild stimulant. Caffeine acts as a central nervous system stimulant in humans and is found in many plants, including coffee and tea plants. It is the world's most widely consumed psychoactive drug; in North America 90% of adults consume it in some form daily. Caffeine helps people fend off fatigue and become more alert.

Caffeine has several health benefits. Researchers have found that it helps increase memory, detoxifies the liver, relieves muscle pain, cleanses the colon, eases depression by increasing dopamine in the brain, and wards off Alzheimer's disease.

There have been many studies done to document the various health benefits that coffee offers its drinkers. Studies suggest that coffee consumption helps reduce the risks of Alzheimer's disease, dementia, Parkinson's disease, heart disease, diabetes mellitus type 2, non-alcoholic fatty liver disease, cirrhosis, and gout. Coffee also has preventative effects against prostate cancer. Coffee also has antioxidants that help prevent free radicals from causing cell damage, which potentially leads to cancer. Habitual coffee consumption has also been found to improve vascular function. The caffeine contained in coffee also helps to alleviate headaches, especially when combined with some form of painkilling medication.

Tea is one of the most consumed beverages in the world. In fact, behind water it is the most consumed beverage in the world. It is consumed more than all other manufactured drinks combined. Tea comes from pouring hot or boiling water over cured tea leaves, which in turn come from tea plants. Tea plants are carefully cultivated both for ease of harvesting and quality control. Tea mainly needs tropical or subtropical climates to grow, however some tea can be grown as far north as Washington State and British Columbia, Canada.

Tea drinking likely began in China during the Shang Dynasty and was used as a medicinal drink. The earliest records of tea drinking in China date to the 10th century BCE. Tea was then introduced to Portuguese priests and merchants during the 16th Century AD. Tea became popular in Britain during the 18th century, but was still rather expensive, leading to a rise in the smuggling of tea in order to make tea more affordable. Tea plants were later introduced to India by the British in an effort to break the Chinese monopoly over tea production.

Tea plays a major role in various cultures throughout the world. The Chinese and Japanese hold tea ceremonies, which is a ritualized way of making and consuming tea. Turkish tea is central to Turkish cuisine. In Arab culture, communal tea consumption is a central social gathering. Sweet tea has become a cultural symbol for the southeastern portion of the United States. Tea even played a major role in the American Revolution in an event now known as the Boston Tea Party. Colonists dumped tea into the Boston Harbor as a way of protesting taxation without representation by the British government.

Tea has many important health benefits to complement its societal and historical benefits. Tea contains many bioactive chemicals, such as flavonoids, amino acids, vitamins, and caffeine. Some research suggests that black and green tea may help to prevent cancer, as well as cardiovascular diseases.

Green tea in particular has been suggested through studies to have protective effects against oral, pharyngeal, oesophageal, prostate, digestive, urinary tract, pancreatic, bladder, skin, lung, colon, breast, and liver cancers. Tea contains caffeine, which is a mild stimulant. Caffeine has several health benefits.

The cannabis plant has many naturally occurring substances that are of great interest in science and medicine. Isolated compounds from the cannabis plant include Δ⁹-tetrahydrocannabinol (THC), cannabidiol (CBD), cannabichromene (CBC), cannabigerol (CBG), cannabidivarin (CBDV), among other compounds. While THC has psychoactive effects, CBD, CBC, CBG, and CBDV do not. Isolated compounds from the cannabis plant are called cannabinoids. There are a total of eighty-five (85) cannabinoids isolated from the cannabis plant. Many researchers have confirmed the medicinal value of cannabinoids. Cannabinoids have been investigated for possible treatment of seizures, nausea, vomiting, lack of appetite, pain, arthritis, inflammation, and other conditions.

The IUPAC nomenclature of THC is (−)-(6aR,10aR)-6,6,9-trimethyl-3-pentyl-6a,7,8,10a-tetrahydro-6H-benzo[c]chromen-1-ol. CBD's IUPAC nomenclature is 2-((1S,6S)-3-methyl-6-(prop-1-en-2-yl)cyclo-hex-2-enyl)-5-pentylbenzene-1,3-diol). CBC has the IUPAC nomenclature of 2-methyl-2-(4-methylpent-3-enyl)-7pentyl-5-chromenol. These are among the most prominent compounds in the family of compounds extracted from the cannabis plant referred to as cannabinoids.

Cannabinoids can be isolated by extraction from cannabis plants. Plants in the cannabis genus include Cannabis sativa, Cannabis ruderalis, and Cannabis indica. These plants are the natural sources of cannabinoids. Cannabinoids are also available in synthetic forms. Methods to synthesize cannabinoids in lab settings were discovered and are practiced currently. Synthetic cannabinoids are more targeted; meaning the synthetic compound usually comes isolated without other cannabinoids mixed in.

Nabilone (racemic(6aR,10aR)-1-hydroxy-6,6-dimethyl-3-(2-methyloctan-2-yl)-7,8,10,10a-tetrahydro-6H-benzo[c]chromen-9(6aH)-one), a synthetic cannabinoid, is believed to have fewer undesired side effects than THC. Nabilone mimics the chemical compound structure of THC. THC also exists in synthetic form under the name dronabinol ((−)-(6aR,10aR)-6,6,9-trimythel-3-pentyl-6a,7,8,10a-tetrahydro-6H-benzo[c]chromen-1-ol)). These synthetic cannabinoids are sold for food supplement purposes and investigated for medicinal purposes. The U.S. Food and Drug Administration approved nabilone for treatment of chemotherapy-induced nausea and vomiting. In the United States, nabilone is marketed under the name Cesamet®.

Cannabis abuse among chronic users is also seen, especially when raw cannabis plant materials are consumed by smoking. Withdrawal symptoms are observed with the discontinuance of cannabis use. Collectively, cannabis abuse is referred to as cannabis use disorder. However, some cannabinoids, such as CBD, may be useful in treating cannabis use disorder.

Cannabidiol from hemp is marketed in the United States as a dietary supplement. Various products containing cannabidiol have been marketed in recent years. Cannabidiol may be consumed by digestion, by inhalation, or by transdermal delivery.

Cannabis and hemp products consumption has many benefits. Cannabis and hemp products are widely used to increase appetite, induce sleep, prevent nausea, and relieve pain, among other beneficial effects. Cannabinoids contained in these products are responsible for these desirable effects. Cannabidiol in particular may induce a calming effect on users. Many ways exist to incorporate cannabinoids into the user's daily routine.

There still exists a demand for a tasty drink with cannabinoid content. A coffee drink containing a desirable amount of cannabinoid gives both alertness and calming effect to the consumer. Cannabis and hemp oil are of a typically strong earthy flavor that affects the coffee's taste. The following invention addresses these problems.

### SUMMARY OF THE INVENTION

The present invention generally relates to cannabinoid caffeinated drinks, methods to make and use of the same. Cannabinoid contents may be added into the coffee or tea drink post brew or pre-brew into the ground coffee powder, onto roasted coffee beans, or on loose tea leaf. An emulsifier or a combination of emulsifiers may be used to successfully incorporate the cannabinoid content. The present invention also discloses appropriate cannabinoid levels to be added into each drink for the best results. Cannabinoid coffee drinks as disclosed in this invention are substantially similar to normal coffee drinks in taste, smell, and appearance, but will enhance the effect of caffeine, making the consumer calm but alert. Tea leaves may be infused with natural cannabinoids by absorbing cannabis oil or hemp oil into an emulsifier before mixing with tea leaves. The cannabinoid tea drink retains the tea taste with a smooth earthy finish.

### ABBREVIATIONS

AD: Anno Domini
BCE: Before Common Era
CBC: Cannabichromene
CBDV: Cannabidivarin
CBD: Cannabidiol
CBG: Cannabigerol
IUPAC: International Union of Pure and Applied Chemistry
MCT: Medium Chain Triglyceride
THC: Tetrahydrocannabinols
wt/wt: weight/weight

### DETAILED DESCRIPTION OF THE INVENTION

This present invention is capable of being embodied in various forms. The description below of several embodiments is made with the understanding that the present disclosure is to be considered as an exemplification of the claimed subject matter, and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in its non-limiting sense to mean that items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more.”

The word “cannabinoid” used in this description, claims, and other conjugations is used to mean any compound that interacts with a cannabinoid receptor and other cannabinoid mimetics, including, but not limited to, certain tetrahydropyran analogs (Δ⁹tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H- dibezo[b,d]pyran-9-ol,(−)-(3S,4S)-7-hydroxy-delta-6-tetrahydrocannabinol-1,1-dimethylheptyl, (+)-(3S,4S)-7-hydroxy-Δ-6-tetrahydrocannabinol, and Δ⁸-tetrahydrocannabinol-11-oic acid); certain piperidine analogs (e.g., (−)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1, 3-benzendi-ol, and 4-(1,1-dimethylheptyl)-2,3′-dihydroxy-6′-α-(3-hydroxypropyl)-1′,-2′,3′,4′,5′,6′-hexahydrobiphenyl), their salts, solvates, metabolites, and metabolic precursors.

The word “cannabidiol” refers to cannabidiol and cannabidiol derivatives. As used in this application, cannabidiol is obtained from industrial hemp extract with trace amount of THC.

The invention is a caffeinated drink with cannabinoids, coffee bean and coffee powder with cannabinoids, or loose tea leaf with cannabinoids. Cannabinoid content is added post-brew into the drinks, or into coffee powder or loose tea leaf, or added during coffee roasting or tea curing process.

In the following embodiments, cannabinoids are obtained from natural sources, namely, by extraction from the plant Cannabis sativa L. Cannabinoids used the following embodiments are derived from natural sources and may carry impurities such as any fatty acids such as linoleic acid and a-linoleic acid, which are natural components of hemp oil or cannabis oil, β-caryophyllene, myrcene, and β-sitosterol. In hemp oil or cannabis oil, cannabinoid content may be by weight up to 50%, up to 45%, up to 40%, up to 35%, up to 30%, up to 25%, up to 20%, up to 15%, up to 10%, or up to 5%.

When the cannabinoid to be used is cannabidiol, cannabidiol may be obtained by cold pressing the seeds and stalks of the plant Cannabis sativa L. Oil obtained from this cold pressing is hemp oil, which is rich in essential fatty acids and cannabidiol. Further processing of hemp oil may increase the cannabidiol content of the processed hemp oil.

Cannabis oil may be obtained by cold pressing and/or extracting the entire Cannabis sativa L. plant. Depending on the specific cultivar, the resulting cannabis oil may contain certain percentages of THC and CBD, as well as other cannabinoids. Other impurities such as linoleic acid and a-linoleic acid, β-caryo phyllene, myrcene, and β-sitosterol may also be present.

Hemp oil or cannabis oil contains Δ⁹-tetrahydrocannabinol, cannabidiol, cannabigerol, among other cannabinoids. Specific weight percentage of each cannabinoid varies, depending on naturally occurring amount and processing methods. To ensure delivery of a specific amount of cannabinoid in each drink, analysis for cannabinoid content is recommended to determine starting amounts of cannabinoids in hemp oil or cannabis oil.

Alternatively, hemp oil or cannabis oil may be distilled to remove the fatty acid content, leaving crystallized cannabinoids in solid form, which may be complexed with randomly methylated β-cyclodextrin to increase solubility in water. Methods to produce this complex are disclosed in Viemstein et. al. Patent Application Publication US2013/0295026. Other methods to complex cannabinoid(s) with various agents to increase solubility may also be available. Crystallized complexed cannabinoids may be ground and added into the drink at desirable concentrations.

In another embodiment, cannabinoids are provided as synthetic cannabinoids. Synthetic cannabinoids are complexed with randomly methylated β-cyclodextrin to increase solubility in water, as described above.

In a preferred embodiment, the invention is a coffee drink with cannabinoids added. Cannabinoids are added together with an emulsifier or a combination of emulsifiers and blended at high velocity for effective dispersion. The resulting combination is a coffee drink infused with cannabinoids while retaining typical coffee taste, smell, and appearance. A mammal, preferably a human being, in consuming this drink, will feel calm and alert.

Coffee is a water-based drink. Hemp oil and cannabis oil both contain a high fatty acid percentage. Emulsifiers are needed to help with dispersion of the hemp oil or cannabis oil used in this embodiment. Effective emulsifiers may be gum arabic, maltodextrin, a-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, and soy lecithin powder.

To add cannabinoids post-brew, a finely dispersed cannabinoid liquid may be made before adding into brewed coffee. To make this dispersed cannabinoid liquid, filtered water is added into a container. Hemp oil or cannabis oil is then added into the water. An emulsifier is then added, such that the emulsifier envelops the oil drops. The composition is then immediately mixed by gentle shaking. High speed stifling or high shear mixing is then introduced until complete dispersion of the emulsion composition is achieved.

To reduce hemp or cannabis oil viscosity, the oil may be combined with other edible oil of lower viscosity, such as olive oil or coconut oil (medium chain triglyceride—MCT). MCT is the preferred oil for combination, as MCT has a lower viscosity than hemp or cannabis oil, does not affect hemp or cannabis taste, and blends well with the coffee or tea drink. The oil blend is mixed with a propeller until uniformed, and cannabinoid content is analyzed to determine cannabinoid content prior to incorporation into the drinks.

In this finely dispersed cannabinoid liquid, water is present in total amount by weight at about 90% to about 98%, about 90% to about 97%, about 90% to about 96%, about 90% to about 95%, about 90% to about 94%, about 90% to about 93%, about 90% to about 92%, about 90% to about 91%.

An emulsifier may be present in a total amount by weight at about 1.5% to about 9.5%, about 1.5% to about 9%, about 1.5% to about 8.5%, about 1.5% to about 8%, about 1.5% to about 7.5%, about 1.5% to about 7%, about 1.5% to about 6.5%, about 1.5% to about 6%, about 1.5% to about 5.5%, about 1.5% to about 5%, about 1.5% to about 4.5%, about 1.5% to about 4%, about 1.5% to about 3.5%, about 1.5% to about 3%, about 1.5% to about 2.5%, or about 1.5% to about 2%.

Hemp oil or cannabis oil may be present in a total amount by weight at about 0.1% to about 3%, about 0.1% to about 2.9%, about 0.1% to about 2.8%, about 0.1% to about 2.7%, about 0.1% to about 2.6%, about 0.1% to about 2.5%, about 0.1% to about 2.4%, about 0.1% to about 2.3%, about 0.1% to about 2.2%, about 0.1% to about 2.1%, about 0.1% to about 2%, about 0.1% to about 1.9%, about 0.1% to about 1.8% about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%.

Alternatively, a cannabis or hemp oil blend with another edible oil may be present in a total amount by weight at about 0.1% to about 5%, at about 0.1% to 4%, at about 0.1% to 3%, at about 0.1% to 2%, at about 0.1% to 1%.

To achieve effective dispersion, the cannabinoid liquid composition is then mixed with a high speed mixer. Preferably the mixer rotates between about 20,000 rpm to about 40,000 rpm. A high shear mixer may also be used in large scale production. For small scale production, a household blender may be able to provide the desirable mixing speed and desirable dispersion of this composition.

Once the cannabinoid liquid composition is uniformly dispersed, it may be added into the coffee drink. The coffee drink may be at a hot or cold temperature, depending on the consumer's need. Once added into the coffee drink, gentle stifling to ensure dispersion in the coffee drink is recommended.

The final cannabinoid coffee drink may contain cannabinoids at between about 10 mg to about 100 mg in an eight ounce drink. The final cannabinoid concentration may be determined by the concentration and quantity of the cannabinoid liquid composition added into the post-brew coffee drink.

In another preferred embodiment, a combination of emulsifiers may be used. Effective combinations of emulsifiers include lecithin (from soy) and xanthan gum, gum arabic and lecithin (from soy), gum arabic and xanthan gum, sodium staeryol lactylate and gum arabic, and mono- and di-glyceride of fatty acids and gum arabic.

In these combinations, the ratio between the emulsifiers is about 1:10 to about 1:1. The two emulsifiers are mixed first according to their respective ratio before being added into the water-oil emulsion and dispersed with a high speed mixer.

In another preferred embodiment, the product is a powder coffee mix comprising cannabinoid. In this embodiment, cannabinoid and an emulsifier or a combination of emulsifiers are introduced into the powdered coffee in solid state.

To obtain the solid mixture of cannabinoid and an emulsifier or a combination or emulsifiers, hemp oil or cannabis oil is mixed with an emulsifier in powder form, preferably gum arabic. A blended cannabinoid oil may also be used for mixing with the powder emulsifier. The amount of oil used may be titrated to ensure the final powder product is not “wet”, such that the powder is still separable into grains. The cannabinoid-emulsifier powder is then mixed with coffee powder.

The resulting mix of cannabinoid and coffee powder may be packaged into a bag for brewing in a coffee brewer. The same cannabinoid coffee powder may also be packaged into a K-cup or a small package for use in one-cup brewing machines.

The efficiency of mixing the cannabinoid-emulsifier(s) solid into coffee powder is approximately 50%. In other words, only approximately 50% of the cannabinoids weight added into coffee powder will be in the brewed coffee drink. Therefore, to achieve a 10 mg-cannabinoid content in a cup of coffee brewed in a K-cup, a cannabinoid-emulsifier solid containing 20 mg of cannabinoids is added into the K-cup.

In another embodiment, the final product is coffee beans coated with cannabinoid. Hemp oil or cannabis oil with cannabinoid content may be added into coffee beans during roasting. Cannabinoid oil coats coffee beans and is carried over once ground. To ensure wider distribution of the cannabinoid oil and easy handling, hemp oil or cannabis oil may be combined with another edible oil, preferably coconut oil or MCT oil to increase the total liquid volume and decrease viscosity.

Cannabinoid delivery efficiency in incorporating cannabinoids into coffee bean during roasting is low. Cannabinoids are lost during roasting, during grinding, and then in the brewing process. The loss rate varies, and it is recommended that post-brew analysis is performed to ascertain the cannabinoid content in the final product.

The coffee drinks or coffee powder may optionally further comprise flavors. Suitable flavors may be chosen from a list comprising of almond, apple cider, apple strudel, apricot, banana, blackberry, brandy, blueberry, bourbon, butternut, pecan, butterscotch, candy corn, caramel, champagne, cherry, cheesecake, chocolate, hazelnut, macadamia, malt, marshmallow, cinnamon, fudge, cocoa, coconut, cookies, cranberry, cream brulee, dulce de leche, eggnog, toffee, espresso, gingerbread, tiramisu, mango, molasses, rum, orange, peppermint, pineapple, pistachio, pumpkin, raspberry, chestnut, sugar, vanilla, maple, among other flavors.

In another embodiment, the invention is a tea drink or tea leaf infused with cannabinoids. In the tea drink, cannabinoids are added with an emulsifier or a combination of emulsifiers to facilitate dispersion. Dissolvable cannabinoids complexed with other compounds may also be added without the use of emulsifiers.

In tea leaf infusion, cannabinoids are absorbed into an emulsifier forming a powder and mixed into the tea leaf preparation. During brewing, the cannabinoid content is released with tea essence and mixed well to give a tea drink. A mammal, preferably a human being, in consuming this drink, will feel calm and alert.

Tea is a water based drink. When hemp oil or cannabis oil is to be infused into tea, an emulsifier or a combination of emulsifiers may be used. Effective emulsifiers are gum arabic, maltodextrin, a-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, or soy lecithin powder.

To prepare a ready-to-drink tea brew, a dispersed solution hemp oil or cannabis oil in water is prepared first. To prepare a cannabinoid water solution, first add hemp or cannabis oil into the emulsifier to form a complex. The oil-emulsifier complex is then added into filtered drinkable water. The solution is blended at high velocity for dispersion.

In the cannabinoid water solution, water is present in a total amount by weight at about 90% to about 98%, about 90% to about 97%, about 90% to about 96%, about 90% to about 95%, about 90% to about 94%, about 90% to about 93%, about 90% to about 92%, about 90% to about 91%.

In a preferred embodiment, gum arabic is used for emulsification. Gum arabic is the most effective emulsifier in water-based solution solutions. Gum arabic may be present in a total amount by weight at about 1.5% to about 9.5%, about 1.5% to about 9%, about 1.5% to about 8.5%, about 1.5% to about 8%, about 1.5% to about 7.5%, about 1.5% to about 7%, about 1.5% to about 6.5%, about 1.5% to about 6%, about 1.5% to about 5.5%, about 1.5% to about 5%, about 1.5% to about 4.5%, about 1.5% to about 4%, about 1.5% to about 3.5%, about 1.5% to about 3%, about 1.5% to about 2.5%, or about 1.5% to about 2%.

Hemp oil or cannabis oil may be present in a total amount by weight at about 0.1% to about 3%, about 0.1% to about 2.9%, about 0.1% to about 2.8%, about 0.1% to about 2.7%, about 0.1% to about 2.6%, about 0.1% to about 2.5%, about 0.1% to about 2.4%, about 0.1% to about 2.3%, about 0.1% to about 2.2%, about 0.1% to about 2.1%, about 0.1% to about 2%, about 0.1% to about 1.9%, about 0.1% to about 1.8% about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%.

After mixing hemp oil or cannabis oil with an emulsifier and adding this complex into water, the cannabinoid liquid composition is then mixed with a high speed mixer. Preferably, the blender blade rotates at between 20,000 rpm to 40,000 rpm for at least 120 seconds.

The cannabinoid liquid composition is then dispersed and may be added into a ready brewed tea drink. The tea drink may be hot or cold, depending on the consumer's need. Gentle stirring to ensure dispersion of the cannabinoid tea drink is recommended. If the ready brewed tea drink is stored in a container for later consumption, the container may be shaken or the liquid may be stirred before use.

Using the appropriate amount of liquid, the final cannabinoid tea brew may contain between 10 mg to 100 mg of cannabinoids in an eight ounce tea drink.

In another preferred embodiment, hemp oil or cannabis oil is mixed with an emulsifier in powder form. The resulting complex in powder form is then mixed with tea leaves at the appropriate ratio. The tea preparation is then brewed in a tea bag or in a tea pot. The resulting tea liquid contains cannabinoids from hemp oil or cannabis oil.

Suitable emulsifiers for use in this preferred embodiment must be in powder form. Examples of suitable emulsifiers are maltodextrin, gum arabic, α-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, or soy lecithin powder. The ratio of hemp oil or cannabis oil to emulsifier is between 1:10 and 1:3.

The loss ratio in brewing the cannabinoid tea leaf preparation is between 50% and 70%. Appropriate measurement is required to determine the resulting amount of cannabinoid in the brewed tea drink.

The hemp oil or cannabis oil and emulsifier complex may also be added into loose tea leaves. The resulting solid mixture may be brewed using a tea bag, a tea pot, or a filter to give a cannabinoid tea drink.

In another preferred embodiment, a combination of emulsifiers may be used. The combination of two or more of maltodextrin, gum arabic, a-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, or soy lecithin powder may be used. The ratio of hemp oil or cannabis oil to the emulsifier mass in combination is between 1:10 to 1:3. Emulsifiers may be combined together in any ratios.

In this preferred embodiment, hemp oil or cannabis oil is combined with the emulsifier mixture and mixed well. When the oil is evenly distributed in the powder mass, the resulting oil-emulsifier powder may be mixed with loose tea leaf. The tea mixture may then be brewed with hot water for consumption.

In another preferred embodiment, tea leaves are cured with cannabis oil or hemp oil. Drops of hemp oil or cannabis oil is added into green tea leaf and mixed well before curing. Loose tea leaf is dried, removing some moisture. Cannabinoids in cannabis oil or hemp oil are infused into tea leaf and will be released when tea is steeped in water.

In the above embodiment, hemp oil or cannabis oil may be mixed with other edible oils, preferably coconut oil, to increase viscosity for better blending. Blended oil is preferred where hemp oil or cannabis oil is used during tea leave curing to infuse cannabinoids.

Tea leaf used in these embodiments may be cured Camellia sinesis plant materials with or without flavors. Tea leaves may be selected from a group comprising of black tea, green tea, oolong tea, matcha tea, white tea, or pu-erh tea, depending on the method of preparation. Herbal teas, or tea preparations derived from other plant materials may also be used. Suitable herbal teas may be selected from a group comprising of rooibos, lemon with ginger, honey bush, spearmint, peppermint, chamomile, tulsi of holy basil, or chamomile with lemongrass.

In another embodiment, yerba mate ground leaf is used in place of Camellia sinesis plant material. Methods to prepare yerba mate drinks with cannabinoids are the same with tea drinks. Alternatively, cannabinoid may also be added into yerba mate dried leaf or added into yerba mater leaf during drying according to the methods above.

Tea preparations in these embodiments may further comprise flavors. Tea flavors are typically created using herbs or plant matters from spice plants. Tea flavors used in these embodiments may be chosen from a group comprising of orange, citrus, rose, jasmine, berry, osmanthus, chrysanthemum, lotus, mint, pandan, smoke, spices (such as ginger, cardamom, cinnamon, cassia, black pepper, clove, anise, fennel, Indian bay leaf, vanilla, nutmeg, or mace), rum, roasted grains and nuts, among other flavors. Flavors may also be added by adding essential oils into loose tea leaf.

Tea leaves infused with the emulsifier—hemp oil mixture may be packaged into tea bags or containers as loose leaf packages for consumption. Tea bags containing this mixture may be brewed using hot water in a cup.

### EXAMPLES

The following examples were carried out using hemp oil containing naturally-occurring CBD, which is derived from industrial hemp cultivars selectively bred for high CBD and low THC content.

### Example 1

The following ingredients are obtained: hemp oil containing 24% (wt/wt) CBD (food grade), gum arabic (natural, food grade) according to the following table:

| Ingredient | Weight (g) |
|---|---|
| Gum arabic | 7 |
| Hemp oil (at 24% CBD by weight) | 1.7 |

Drop hemp oil into the gum arabic mass drop-by-drop, then mix gently using a spatula. After the gum arabic—hemp oil complex is evenly mixed, weight 0.75 gram of this mixture into a container. Then, add 4 grams of black tea leaf. Mix the tea leaf—gum arabic—hemp oil mixture evenly in the container using the spatula.

Boil water to 100° C. Using two cups of water and a tea pot, brew the above tea leaf—gum arabic—hemp oil mixture. The resulting tea liquid is then served. Due to loss in the brewing process, each tea cup now contains approximately 10 mg of CBD as a natural constituent of hemp oil.

### Example 2

The following ingredients are obtained: water, hemp oil with 15% (wt/wt) CBD content, gum arabic (natural) according to the following table:

| Ingredient | Weight | % (wt/wt) |
|---|---|---|
| Water | 194 | 97 |
| Gum arabic | 5 | 2.5 |
| Hemp oil (at 15% CBD by weight) | 1 | 0.5 |
| Total | 200 | 100 |

Add 194 grams of filtered water into a beaker. Using a pipette, add 1 gram of hemp oil (with 15% by weight CBD) into the beaker. Weigh and add 5 grams of gum arabic, take care to add gum arabic on top of the hemp oil drops.

Pour the entire mixture into a blender and blend for 1 minute. The resulting liquid composition is a finely dispersed liquid with a yellowish white color. This is the cannabinoid liquid composition.

Add 20 grams of this liquid into an 8-ounce coffee cup and stir gently. The coffee cup now has 15 mg of cannabidiol.

All references, including publications, patent applications, and patents, cited herein are hereby incorporated by reference to the same extent as if each reference were individually and specifically indicated to be incorporated by reference and were set forth in its entirety herein.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

We claim:

1. A caffeinated drink with at least one cannabinoid.

2. The caffeinated drink of claim 1, wherein the drink is coffee.

3. The caffeinated drink of claim 1, where in the drink is tea.

4. The caffeinated drink of claim 1, wherein the drink is yerba mate.

5. The caffeinated drink of claim 1, wherein the at least one cannabinoid is Δ⁹-tetrahydrocannabinol.

6. The caffeinated drink of claim 1, wherein the at least one cannabinoid is cannabidiol.

7. The caffeinated drink of claim 1, wherein the at least one cannabinoid is cannabigerol.

8. The caffeinated drink of claim 1, wherein the at least one cannabinoid is from natural sources.

9. The caffeinated drink of claim 1, wherein the at least one cannabinoid is synthetic.

10. The caffeinated drink of claim 1, wherein the at least one cannabinoid is provided as hemp oil or cannabis oil.

11. The caffeinated drink of claim 1, wherein the at least one cannabinoid is complexed with randomly methylated β-cyclodextrin.

12. The caffeinated drink of claim 1, further comprising at least one emulsifier.

13. The caffeinated drink of claim 12, wherein the at least one emulsifier is selected from a group comprising of gum arabic, maltodextrin, α-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, and soy lecithin powder.

14. The caffeinated drink of claim 12, further comprising at least one flavor enhancer.

15. The caffeinated drink of claim 14, wherein the at least one flavor enhancer is selected from a group comprising of orange, citrus, rose, jasmine, berry, osmanthus, chrysanthemum, lotus, mint, pandan, smoke, ginger, cardamom, cinnamon, cassia, black pepper, clove, anise, fennel, Indian bay leaf, vanilla, nutmeg, mace, rum, roasted grains, almond, apple cider, apple strudel, apricot, banana, blackberry, brandy, blueberry, bourbon, butternut, pecan, butterscotch, candy corn, caramel, champagne, cherry, cheesecake, chocolate, hazelnut, macadamia, malt, marshmallow, fudge, cocoa, coconut, cookies, cranberry, cream brulee, dulce de leche, eggnog, toffee, espresso, gingerbread, tiramisu, mango, molasses, rum, peppermint, pineapple, pistachio, pumpkin, raspberry, chestnut, sugar, and maple.

16. A cannabinoid coffee powder comprising coffee powder and at least one cannabinoid.

17. The coffee powder of claim 16, wherein the at least one cannabinoid is natural.

18. The coffee powder of claim 16, wherein the at least one cannabinoid is provided as hemp oil or cannabis oil.

19. The coffee powder of claim 16, wherein the at least one cannabinoid is complex with randomly methylated β-cyclodextrin.

20. The coffee powder of claim 16, further comprising at least one emulsifier.

21. The coffee powder of claim 20, wherein the at least one emulsifier is selected from a group comprising of gum arabic, maltodextrin, a-cyclodextrin, lambda or kappa carageenan, iota carrageenan, sodium caseinate, food grade polysorbate, glycerol monostearate, guar gum, disodium phospate dihydrate, propylene glycol alginate, sodium stearyol lactylate, and soy lecithin powder.

22. The coffee powder of claim 21, further comprising at least one flavor.

23. The coffee powder of claim 22, wherein the at least one flavor enhancer is selected from a group comprising of orange, citrus, rose, jasmine, berry, osmanthus, chrysanthemum, lotus, mint, pandan, smoke, ginger, cardamom, cinnamon, cassia, black pepper, clove, anise, fennel, Indian bay leaf, vanilla, nutmeg, mace, rum, roasted grains, almond, apple cider, apple strudel, apricot, banana, blackberry, brandy, blueberry, bourbon, butternut, pecan, butterscotch, candy corn, caramel, champagne, cherry, cheesecake, chocolate, hazelnut, macadamia, malt, marshmallow, fudge, cocoa, coconut, cookies, cranberry, cream brulee, dulce de leche, eggnog, toffee, espresso, gingerbread, tiramisu, mango, molasses, rum, peppermint, pineapple, pistachio, pumpkin, raspberry, chestnut, sugar, and maple.

24. A cannabinoid loose tea leaf composition comprising tea leaf and at least one cannabinoid.

25. The cannabinoid loose tea leaf composition of claim 24, wherein the tea leaf is selected from a group comprising of black tea, green tea, oolong tea, matcha tea, white tea, and pu-erh tea.

26. The cannabinoid loose tea leaf of claim 24, wherein the at least one cannabinoid is natural.

27. The cannabinoid loose tea leaf of claim 24, wherein the at least one cannabinoid is provided as hemp oil or cannabis oil.

28. The cannabinoid loose tea leaf of claim 24, further comprising at least one flavor.

29. The cannabinoid loose tea leaf of claim 28, wherein the at least one flavor is selected from a group comprising of orange, citrus, rose, jasmine, berry, osmanthus, chrysanthemum, lotus, mint, pandan, smoke, ginger, cardamom, cinnamon, cassia, black pepper, clove, anise, fennel, Indian bay leaf, vanilla, nutmeg, mace, rum, roasted grains, and nuts.

30. A method to make a caffeinated cannabinoid drink comprising the steps of:
(a) mixing at least one emulsifier with at least one cannabinoid;
(b) adding the mixture in step (a) into water; and
(c) blending the solution at high speed.

31. A method to make a cannabinoid coffee powder comprising the steps of:
(a) mixing at least one cannabinoid with at least one emulsifier in powder form; and
(b) combining the mixture in (a) with coffee powder.

32. A method to make a cannabinoid loose tea leaf composition comprising the steps of:
(a) mixing at least one cannabinoid with at least one emulsifier in powder form; and
(b) combining the mixture in step (a) with loose leaf tea.

33. A method to make a cannabinoid loose tea leaf composition comprising the steps of:
(a) mixing hemp oil or cannabis oil with naturally occurring cannabinoid and green tea leaf; and
(b) curing the tea leaf.
